(19) United States
(12) Patent Application Publication
(10) Pub. No.: US 2006/0182789 A1
(43) Pub. Date: Aug. 17, 2006

(54) Apparatus and method for transdermal delivery of epoetin-based agents

(76) Inventors: Mahmoud Ameri, Fremont, CA (US); Michel J.N. Cormier, Mountain View, CA (US); Yuh-Fun Maa, Millbrae, CA (US); Peter Daddona, Menlo Park, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/355,856

(22) Filed: Feb. 15, 2006

Related U.S. Application Data
(60) Provisional application No. 60/653,676, filed on Feb. 16, 2005.

(51) Int. Cl. A61K 38/18; A61F 13/02; A61M 31/00

(52) U.S. Cl. 424/448; 514/12; 604/500

(57) ABSTRACT

An apparatus and method for transdermally delivering a biologically active agent comprising a delivery system having a microprojection member (or system) that includes a plurality of microprojections (or array thereof) that are adapted to pierce through the stratum corneum into the underlying epidermis layer, or epidermis and dermis layers. In one embodiment, an Epoetin-based agent is contained in a biocompatible coating that is applied to the microprojection member. In a further embodiment, the delivery system includes a gel pack having an Epoetin-based agent-containing hydrogel formulation that is disposed on the microprojection member after application to the skin of a patient. In an alternative embodiment, the Epoetin-based agent is contained in both the coating and the hydrogel formulation.

### CROSS-REFERENCE TO RELATED APPLICATIONS

This application claims the benefit of U.S. Provisional Application No. 60/653,676, filed Feb. 16, 2005.

### FIELD OF THE PRESENT INVENTION

The present invention relates generally to transdermal agent delivery systems and methods. More particularly, the invention relates to an apparatus and method for transdermal delivery of Epoetin-based agents.

### BACKGROUND OF THE INVENTION

Active agents (or drugs) are most typically administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure, which sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and devices developed to mechanically penetrate or disrupt the outermost skin layers thereby creating pathways into the skin in order to enhance the amount of agent being transdermally delivered. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

Epoetin and its salts (i.e., Epoetin-based agents) are typically administered to treat anemia caused by the failure of the kidneys to produce sufficient erythropoietin (EPO) (a hormone that stimulates red blood cell production.) Epoetin-based agents are also administered to treat anemia associated with conditions, such as cancer and HIV/AIDS, or following surgery or chemotherapy. In addition, EPO has shown activity in the treatment of the tumor-associated anemia and for correction of tumor hypoxia. Recent work suggests that EPO treatment may be beneficial for patients with (chronic) infections (HIV, inflammatory bowel disease, septic episodes) and for treatment of the fatigue syndrome following cancer chemotherapy. In addition, EPO may also improve stem cell engraftment following high-dose chemotherapy and can increase survival rates of patients with aplastic anemia and myelodysplastic syndrome.

EPO is both hematopoietic and tissue protective, putatively through interaction with different receptors. Carbamylated EPO (CEPO) or certain EPO mutants do not bind to the classical EPO receptor and do not show any hematopoietic activity. Nevertheless, CEPO and various nonhematopoietic mutants are cytoprotective in vitro and confer neuroprotection against stroke, spinal cord compression, diabetic neuropathy, and experimental autoimmune encephalomyelitis at a potency and efficacy comparable to EPO.

EPO is produced in the kidney and stimulates the division and differentiation of committed erythroid progenitors in the bone marrow. The typical therapeutic form of Epotein is Epoetin-alpha, a 165 amino acid glycoprotein manufactured by recombinant DNA technology. Epoetin-alpha exhibits the same biological effects as endogenous EPO.

At present, Epoetin alpha is only administered by injection. As stated, the direct injection of an agent into the bloodstream is often inconvenient and painful, which often results in poor patient compliance. Intracutaneous administration of an Epoetin-based agent is thus likely to increase patient compliance and improve patient acceptance of the agent, which typically must be administered daily.

It would therefore be desirable to provide an agent delivery system that facilitates intracutaneous administration of Epoetin-based agents.

It is therefore an object of the present invention to provide a transdermal agent delivery apparatus and method that provides intracutaneous delivery of an Epoetin-based agent to a patient.

It is another object of the invention to provide an Epoetin-based agent formulation for intracutaneous delivery to a patient.

It is another object of the present invention to provide a transdermal agent delivery apparatus and method that includes microprojections coated with a biocompatible coating that includes at least one biologically active agent, preferably, an Epoetin-based agent, more preferably, an Epotein alpha-based agent.

It is yet another object of the present invention to provide a transdermal agent delivery apparatus and method that includes a gel pack adapted to receive a hydrogel formulation that contains an Epoetin-based agent.

### SUMMARY OF THE INVENTION

In accordance with the above objects and those that will be mentioned and will become apparent below, the apparatus and method for transdermally delivering an Epoetin-based agent in accordance with this invention generally comprises a delivery system having a microprojection member (or system) that includes a plurality of microprojections (or array thereof) that are adapted to pierce through the stratum corneum into the underlying epidermis layer, or epidermis and dermis layers. In one embodiment, the microprojection member includes a biocompatible coating having at least one Epoetin-based agent disposed therein.

In one embodiment of the invention, the microprojection member includes a microprojection array having a microprojection density of at least approximately 100 microprojections/cm², more preferably, a density in the range of at least approximately 200-3000 microprojections/cm².

Preferably, the length of each microprojection is less than 1000 microns, more preferably, less than 500 microns. In a preferred embodiment of the invention, the length of each microprojection is in the range of approximately 50-145 microns.

In one embodiment, the microprojection member is constructed out of stainless steel, titanium, nickel titanium alloys, or similar biocompatible materials, such as polymeric materials.

In another embodiment, the microprojection member is constructed out of a non-conductive material, such as a polymer. Alternatively, the microprojection member can be coated with a non-conductive material, such as Parylene®, or a hydrophobic material, such as Teflon®, silicon or other low energy material.

The coating formulations applied to the microprojection member to form solid biocompatible coatings can comprise aqueous and non-aqueous formulations having at least one Epoetin-based agent, which can be dissolved within a biocompatible carrier or suspended within the carrier.

In a preferred embodiment, the Epoetin-based agent is selected from the group consisting of Epoetin alpha, Epoetin beta, darbepoetin alfa, pharmaceutically acceptable salts, analogs, and simple derivatives thereof, closely related molecules, and mixtures thereof. The most preferred Epoetin-based agent comprises Epoetin alpha.

Suitable Epoetin-based salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, 2-hydroxyisobutyrate, lactate, malate, pyruvate, fumarate, tartarate, nitrate, phosphate, benzene, sulfonate, methane sulfonate, sulfate and sulfonate.

Suitable simple Epoetin-based derivatives and closely related molecules include, without limitation, pegylated derivatives, carbamylated derivatives, glycosylated derivatives, fusion derivatives, EPO muteins, nonhematopoietic mutants, and thrombopoietin.

In one embodiment of the invention, the Epoetin-based agent comprises in the range of approximately 1-30 wt. % of the coating formulation.

Preferably, the coating on the microprojection member includes a total dose of Epoetin-based agent in the range of 15-200 μg.

Preferably, the pH of the coating formulation is below approximately pH 4.5, or above approximately pH 5.0. More preferably, the pH of the coating formulation is in the range of approximately pH 2 to 4.5, or in the range of approximately pH 5.0 to pH 11. Even more preferably, the pH of the coating formulation is in the range of approximately pH 2 to pH 4 or in the range of approximately pH 5.5 to pH 9.5.

A counterion can be present in the coating formulation in an amount necessary to neutralize the charge present on the Epoetin-based agent at the pH of the formulation. Below a pH of about 4.8, the Epoetin-based agent will bear a positive charge. Therefore, an acidic counterion is utilized. Excess of counterion (as the free acid or as a salt) can be added in order to control pH and to provide adequate buffering capacity.

At a pH above about 4.8, Epoetin alpha will bear a negative charge. Therefore, a basic counterion is utilized. Excess of counterion (as the free base or as a salt) can similarly be added in order to control pH and to provide adequate buffering capacity.

In one embodiment of the invention, the acidic counterion comprises a non-volatile weak acid. Non-volatile weak acid counterions are defined as weak acids presenting at least one acidic pKa and a melting point higher than about 50° C. or a boiling point higher than about 170° C. at Pₐₜₘ. Examples of such acids include citric acid, succinic acid, glycolic acid, lactic acid, malic acid, pyruvic acid, tartaric acid, tartronic acid and fumaric acid.

In another embodiment of the invention, the counterion comprises a strong acid. Strong acids are defined as presenting at least one pKa lower than about 2. Examples of such acids include hydrochloric acid, hydrobromic acid, nitric acid, sulfuric acid, maleic acid, phosphoric acid, benzene sulfonic acid and methane sulfonic acid.

In another embodiment of the invention, the counterion comprises a mixture of counterions, wherein at least one of the counterions comprises a strong acid and at least one of the counterions comprises a non-volatile weak acid.

In yet another embodiment of the invention, the counterion comprises a mixture of counterions, wherein at least one of the counterions comprises a strong acid and at least one of the counterions comprises a weak acid with high volatility. Highly volatile weak acid counterions are defined as weak acids presenting at least one pKa higher than about 2 and having a melting point lower than about 50° C. or a boiling point lower than about 170° C. at Pₐₜₘ. Examples of such acids include acetic acid, propionic acid, pentanoic acid and the like.

In another embodiment of the invention, the basic counterion comprises a weak base with low volatility. Low volatility weak base counterions are defined as weak bases presenting at least one basic pKa and a melting point higher than about 50° C. or a boiling point higher than about 170° C. at Pₐₜₘ. Examples of such bases include monoethanolomine, diethanolamine, triethanolamine, tromethamine, methylglucamine and glucosamine.

In another embodiment of the invention, the counterion comprises a strong base presenting at least one pKa higher than about 12. Examples of such bases include sodium hydroxide, potassium hydroxide, calcium hydroxide and magnesium hydroxide.

Another embodiment of the invention is directed to a mixture of counterions, wherein at least one of the counterions comprises a strong base and at least one of the counterions comprises a weak base with low volatility.

Another embodiment of the invention is directed to a mixture of counterions, wherein at least one of the counterions comprises a strong base and at least one of the counterions comprises a weak base with high volatility. Highly volatile weak base counterions are defined as weak bases presenting at least one pKa lower than about 12 and a melting point lower than about 50° C. or a boiling point lower than about 170° C. at Pₐₜₘ. Examples of such bases include ammonia and morpholine.

In one embodiment of the invention, the coating formulation includes at least one buffer. Examples of suitable buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols, such as laureth-4.

In one embodiment of the invention, the concentration of the surfactant is in the range of approximately 0.001-2 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropylcellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hyroxyethyl starch, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 0.01-20 wt. %, more preferably, in the range of approximately 0.03-10 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar. Suitable non-reducing sugars include, for example, sucrose, trehalose, stachyose, or raffinose. Suitable polysaccharides include, for example, dextran, soluble starch, dextrin, and inulin. Suitable reducing sugars include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In one embodiment of the invention, the coating formulation includes an antioxidant, which can comprise, without limitation, sequestering agents, such sodium citrate, citric acid, EDTA (ethylenedinitrilo-tetraacetic acid) or free radical scavengers, such as ascorbic acid, methionine, sodiumascorbate, and the like.

In yet another embodiment of the invention, the coating formulation includes a solubilizing/complexing agent, which can comprise, without limitation, Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilizing/complexing agents are beta-cyclodextrin, hydroxypropyl beta-cyclodextrin, 2-hydroxypropyl-beta-Cyclodextrin and sulfobutylether7 beta-cyclodextrin.

The concentration of the solubilizing/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt. % to 50 wt. % of the coating formulation.

Preferably, the coating formulations have a viscosity less than approximately 500 centipoise and greater than approximately 3 centipose.

In one embodiment of the invention, the thickness of the biocompatible coating is less than 25 microns, more preferably, less than 10 microns, as measured from the microprojection surface.

In a further embodiment of the invention, the delivery system includes a gel pack, the gel pack being adapted to receive a hydrogel formulation.

In at least one embodiment of the invention, the hydrogel formulation contains at least one Epoetin-based agent.

Preferably, the Epoetin-based agent is present in the hydrogel formulation at a concentration in the range of approximately 0.1-2 wt. % of the hydrogel formulation.

Preferably, the pH of the hydrogel formulation is below approximately pH 4.5, or above approximately pH 5.0. More preferably, the pH of the hydrogel formulation is in the range of approximately pH 2 to pH 4.5, or in the range of approximately pH 5.0 to pH 11. Even more preferably, the pH of the hydrogel formulation is in the range of approximately pH 2 to pH 4, or in the range of approximately pH 5.5 to pH 9.5.

A counterion can similarly be present in the hydrogel formulation in an amount necessary to neutralize the charge present on the Epoetin-based agent at the pH of the hydrogel formulation. As stated above, below a pH of about 4.8, the Epoetin-based agent will bear a positive charge. Therefore an acidic counterion is utilized.

Above a pH of 4.8, Epoetin alpha will bear a negative charge. Therefore a basic counterion is utilized.

Excess of counterion (as the free base or as a salt) can similarly be added to the hydrogel formulation in order to control pH and to provide adequate buffering capacity.

The hydrogel formulation(s) contained in the gel pack preferably comprise water-based hydrogels having macromolecular polymeric networks.

In a preferred embodiment of the invention, the polymer network comprises, without limitation, hyroxyethyl starch, dextran, hydroxyethylcellulose (HEC), hydroxypropylmethylcellulose (HPMC), hydroxypropycellulose (HPC), methylcellulose (MC), hydroxyethyl-methylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

The hydrogel formulation preferably includes at least one of the aforementioned surfactants.

In one embodiment of the invention, the hydrogel formulation includes at least one of the aforementioned buffers.

In another embodiment, the hydrogel formulation includes at least one of the aforementioned polymeric materials or polymers having amphiphilic properties.

In a further embodiment of the invention, the hydrogel formulation includes at least one of the aforementioned solubilizing/complexing agents.

In a further embodiment of the invention, the hydrogel formulation includes at least one of the aforementioned pathway patency modulators.

In yet another embodiment of the invention, the hydrogel formulation includes at least one of the aforementioned vasoconstrictors.

In another embodiment, the hydrogel formulation includes at least one of the aforementioned stabilizing agents.

In one embodiment of the invention, the hydrogel formulation includes at least one of the aforementioned antioxidants.

In another embodiment of the invention, the hydrogel formulation includes at least one non-aqueous solvent, such as ethanol, isopropanol, methanol, propanol, butanol, propylene glycol, dimethyl sulphoxide and polyethylene glycol 400. Preferably, the non-aqueous solvent is present in the range of approximately 1 wt. % to 50 wt. % of the hydrogel formulation.

In accordance with yet another embodiment of the invention, the delivery system includes (i) a gel pack containing a hydrogel formulation and (ii) a microprojection member having top and bottom surfaces, a plurality of openings that extend through the microprojection member and a plurality of stratum corneum-piercing microprotrusions that project from the bottom surface of the microprojection member, the microprojection member including a solid film having at least one Epoetin-based agent. In one embodiment, the solid film is disposed proximate the top surface of the microprojection member. In another embodiment, the solid film is disposed proximate the bottom surface of the microprojection member.

In one embodiment of the invention, the hydrogel formulation includes at least one Epoetin-based agent.

In another embodiment, the hydrogel formulation is devoid of an Epoetin-based agent.

In one embodiment, the solid film is made by casting a liquid formulation consisting of the Epoetin-based agent, a polymeric material, such as hyroxyethyl starch, dextran, hydroxyethylcellulose (HEC), hydroxypropylmethylcellulose (HPMC), hydroxypropylcellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone), or pluronics, a plasticising agent, such as glycerol, propylene glycol, or polyethylene glycol, a surfactant, such as Tween 20 or Tween 80, and a volatile solvent, such as water, isopropanol, methanol or ethanol.

In one embodiment, the liquid formulation used to produce the solid film comprises 0.1-20 wt. % Epoetin-based agent, 5-40 wt. % polymer, 5-40 wt. % plasticiser, 0-2 wt. % surfactant, and the balance of volatile solvent. Following casting and subsequent evaporation of the solvent, a solid film is produced.

Preferably, the Epoetin-based agent is present in the liquid formulation used to produce the solid film at a concentration in the range of approximately 0.1 to 20 wt. %.

Preferably, the pH of the liquid formulation used to produce the solid film is below approximately pH 4.5 or above approximately pH 5.0. More preferably, the pH of the formulation used to produce the solid film is in the range of approximately pH 2 to pH 4.5, or in the range of approximately pH 5.0 to pH 11. Even more preferably, the pH of the liquid formulation used to produce the solid film is in the range of approximately pH 2 to pH 4, or in the range of approximately pH 5.5 to pH 9.5.

In one embodiment of the invention, the liquid formulation used to produce the solid film includes at least one of the aforementioned counterions or a mixture thereof.

In another embodiment, the liquid formulation used to produce the solid film includes at least one of the aforementioned stabilizing agents.

In one embodiment of the invention, the liquid formulation used to produce the solid film includes at least one of the aforementioned buffers.

In another embodiment of the invention, the liquid formulation used to produce the solid film includes at least one of the aforementioned solubilizing/complexing agents.

In a further embodiment of the invention, the liquid formulation used to produce the solid film includes at least one of the aforementioned vasoconstrictors.

In a further embodiment of the invention, the liquid formulation used to produce the solid film includes at least one of the aforementioned pathway patency modulators.

In one embodiment of the invention, the liquid formulation used to produce the solid film includes at least one of the aforementioned antioxidants.

In accordance with one embodiment of the invention, the method for delivering an Epoetin-based agent to a patient includes the following steps: (i) providing a microprojection member having a biocompatible coating that includes at least one Epoetin-based agent and (ii) applying the coated microprojection member to the patient's skin, wherein the microprojections pierce the stratum corneum. The coated microprojection member is preferably left on the skin for a period lasting from 5 seconds to 24 hours. Following the desired wearing time, the microprojection member is removed.

In accordance with a further embodiment of the invention, the method for delivering an Epoetin-based agent to a patient includes the following steps: (i) providing a microprojection member having a solid film disposed proximate to (or on) the member, the film including at least one Epoetin-based agent and (ii) applying the microprojection member to the patient's skin, wherein the microprojections pierce the stratum corneum. The microprojection member is preferably left on the skin for a period lasting from 5 minutes to 24 hours. Following the desired wearing time, the microprojection member is removed.

In a further embodiment of the invention, the microprojection member includes a gel pack having an Epoetin-based agent-containing hydrogel formulation and after the microprojection member is applied to the patient's skin, the gel pack is placed on top of the applied member, wherein the hydrogel formulation migrates into and through the microslits in the stratum corneum produced by the microprojections. The microprojection member-gel pack assembly is preferably left on the skin for a period lasting from 5 minutes to 24 hours. Following the desired wearing time, the microprojection member and gel pack are removed.

In a further aspect of the gel pack embodiment, the Epoetin-based agent is contained in a solid film and the hydrogel formulation is devoid of an Epoetin-based agent and, hence, is merely a hydration mechanism.

In another embodiment of the invention, the microprojection device is applied to the patient's skin and immediately removed. The gel pack having an Epoetin-based based agent-containing hydrogel formulation is then placed on top of the pretreated skin, wherein the hydrogel formulation migrates into and through the microslits in the stratum corneum produced by the microprojections. Preferably, the gel pack is left on the skin for a period lasting from 5 minutes to 24 hours. Following the desired wearing time, the gel pack is removed.

In yet another embodiment of the invention, the microprojection member having an Epoetin-based agent-containing biocompatible coating is applied to the patient's skin, the gel pack having an Epoetin-based agent-containing hydrogel formulation is then placed on top of the applied microprojection member, wherein the hydrogel formulation migrates into and through the microslits in the stratum corneum produced by the microprojections. The microprojection member-gel pack assembly is preferably left on the skin for a period lasting in the range of approximately 5 minutes to 24 hours. Following the desired wearing time, the microprojection member and gel pack are removed.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member;

FIG. 2 is a perspective view of the microprojection member shown in FIG. 1 having a coating deposited on the microprojections, according to the invention;

FIG. 3 is a side sectional view of a microprojection member having an adhesive backing;

FIG. 4 is an exploded perspective view of one embodiment of a gel pack of a microprojection system;

FIG. 5 is an exploded perspective view of one embodiment of a microprojection member of a microprojection system;

FIG. 6 is a perspective view of one embodiment of a microprojection assembly comprising the gel pack shown in FIG. 4 and the microprojection member shown in FIG. 5;

FIG. 7 is a side sectional view of a retainer having a microprojection member disposed therein;

FIG. 8 is a perspective view of the retainer shown in FIG. 7;

FIG. 9 is an exploded perspective view of an applicator and retainer;

FIG. 10 is a graph illustrating the predicted charge profile for an Epoetin-based agent; and

FIG. 11 is a graph illustrating the predicted mole ratios of the net-charged species of an Epoetin-based agent.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### DEFINITIONS

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the Epoetin-based agent is delivered, before and during transdermal flux of the Epoetin-based agent, during transdermal flux of the Epoetin-based agent, during and after transdermal flux of the Epoetin-based agent, and/or after transdermal flux of the Epoetin-based agent. Additionally, two or more Epoetin-based agents can be formulated in the coatings and/or hydrogel formulation, resulting in co-delivery of the Epoetin-based agents.

The term “Epoetin-based agent”, as used herein, includes, without limitation, recombinant Epoetin alpha, synthetic Epotein alpha, Epoetin alpha salts, analogs and simple derivatives of Epoetin alpha, recombinant Epoetin beta, synthetic Epotein beta, Epoetin beta salts, simple derivatives and analogs of Epoetin beta, darbepoetin alfa, recombinant darbepoetin alfa, synthetic darbepoetin alfa, darbepoetin alfa salts, simple derivatives and analogs of darbepoetin alfa, and closely related molecules to any of the foregoing.

Examples of pharmaceutically acceptable Epoetin-based salts include, without limitation, acetate, propionate, butyrate, pentanoate, hexanoate, heptanoate, levulinate, chloride, bromide, citrate, succinate, maleate, glycolate gluconate, glucuronate, 3-hydroxyisobutrate, 2-hydroxyisobutyrate, lactate, malate, pyruvate, fumarate, tartarate, tartronate, nitrate, phosphate, benzene sulfonate, methane sulfonate, sulfate, sulfonate, tricarballylicate, malonate, adipate, citraconate, glutarate, itaconate, mesaconate, citramalate, dimethylolpropionate, tiglicate, glycerate, methacrylate, isocrotonate, β-hydroxybutyrate, crotonate, angelate, hydracrylate, ascorbate, aspartate, glutamate.

Examples of simple Epoetin-based derivatives and closely related molecules include, without limitation, pegylated derivatives, carbamylated derivatives, glycosylated derivatives, fusion derivatives, EPO muteins, nonhematopoietic mutants, and thrombopoietin.

The noted Epoetin-based agents can also be in various forms, such as free bases or acids, charged or uncharged molecules, components of molecular complexes or nonirritating, pharmacologically acceptable salts.

It is to be understood that more than one Epoetin-based agent can be incorporated into the agent source, reservoirs, and/or coatings of this invention, and that the use of the term “Epoetin-based agent” in no way excludes the use of two or more such active agents or drugs.

The term “microprojections”, as used herein, refers to piercing elements that are adapted to pierce or cut through the stratum corneum into the underlying epidermis layer, or epidermis and dermis layers, of the skin of a living animal, particularly a mammal and more particularly a human.

In one embodiment of the invention, the microprojections have a projection length less than about 1000 microns, more preferably, less than 500 microns. In a preferred embodiment, the microprojections have a length less than about 145 microns, more preferably, the length is in the range of about 50-145 microns, even more preferably, in a range of about 70-140 microns.

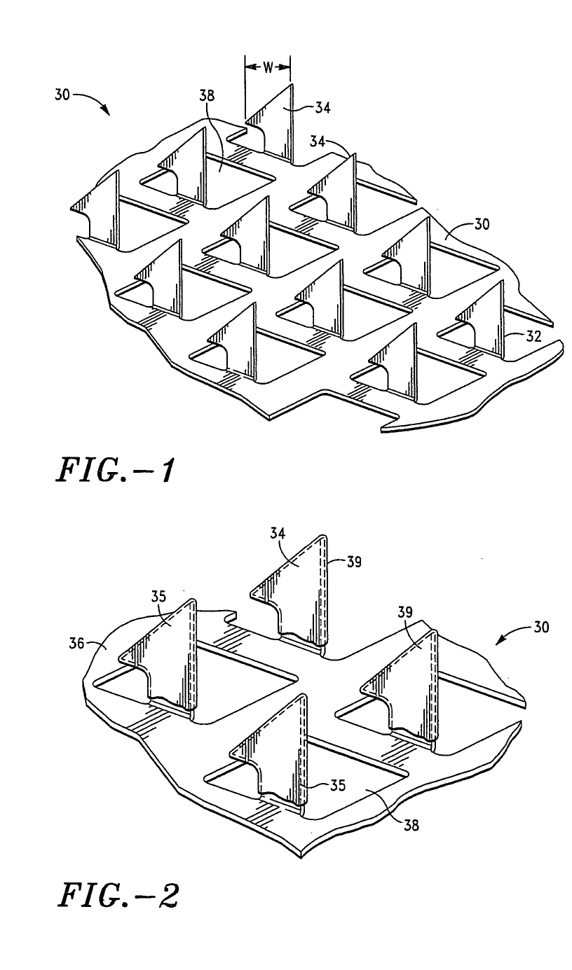

The microprojections preferably have a width (designated “W” in FIG. 1) in the range of approximately 5-50 microns and a thickness in the range of approximately 5-50 microns.

The microprojections of the invention can be formed in different shapes, such as needles, blades, pins, punches, and combinations thereof.

The term “microprojection member”, as used herein, generally connotes a base member having a plurality of microprojections arranged in an array. The microprojection member can be formed by etching or punching a plurality of microprojections from a thin sheet and folding or bending the microprojections out of the plane of the sheet to form a configuration, such as that shown in FIG. 1.

The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture having at least one Epoetin-based agent that is employed to coat the microprojections and/or arrays thereof. The Epoetin-based agent can be in solution or suspension in the formulation.

The term “biocompatible coating” and “solid coating”, as used herein, is meant to mean and include a “coating formulation” in a substantially solid state.

As indicated above, the present invention generally comprises a delivery system including microprojection member (or system) having a plurality of microprojections (or array thereof) that are adapted to pierce through the stratum corneum into the underlying epidermis layer, or epidermis and dermis layers.

In one embodiment, the microprojections have a biocompatible coating thereon that contains at least one Epoetin-based agent. Upon piercing the stratum corneum layer of the skin, the agent-containing coating is dissolved by body fluid (intracellular fluids and extracellular fluids such as interstitial fluid) and released into the skin (i.e., bolus delivery) for systemic therapy. Preferably, the total dose of Epoetin-based agent delivered intracutaneously per administration is in the range of approximately 10-200 μg. More preferably, the total dose of Epoetin-based agent delivered intracutaneously per administration is in the range of about 15-150 μg delivered once every two weeks and up to once a day.

According to the invention, the delivery system of the invention is particularly suitable for administration of therapeutic agents that would otherwise need to be delivered via hypodermic injection or intravenous infusion. The delivery system of the present invention is relatively simple, convenient and virtually painless. The use of the delivery system is thus likely to increase patient compliance.

Referring now to FIG. 1, there is shown one embodiment of a microprojection member 30 for use with the present invention. As illustrated in FIG. 1, the microprojection member 30 includes a microprojection array 32 having a plurality of microprojections 34. The microprojections 34 preferably extend at substantially a 90° angle from a sheet 36, which in the noted embodiment includes openings 38.

According to the invention, the sheet 36 can be incorporated into a delivery patch, including a backing 40 for the sheet 36, and can additionally include adhesive 16 for adhering the patch to the skin (see FIG. 3). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 100 microprojections/cm², more preferably, the density is in the range of approximately 200-3000 microprojections/cm². Preferably, the number of openings per unit area through which the agent passes is at least in the range of approximately 10-2000 openings/cm².

As indicated, the microprojections 34 preferably have a length less than 1000 microns, more preferably, less than 500 microns. In one embodiment, the microprojections have a length in the range of approximately 50-145 microns, more preferably, in the range of approximately 70-140 microns. The microprojections 34 also preferably have a width and thickness in the range of approximately 5-50 microns.

A preferred embodiment of a microprojection array is disclosed in U.S. Application No. 60/653,675, filed Feb. 16, 2005, the disclosure of which is incorporated herein by reference.

The microprojection member 30 can be manufactured from various metals, such as stainless steel, titanium, nickel titanium alloys, or similar biocompatible materials, such as polymeric materials.

According to the invention, the microprojection member 30 can also be constructed out of a non-conductive material, such as a polymer. Alternatively, the microprojection member can be coated with a non-conductive material, such as Parylene®, or a hydrophobic material, such as Teflon®, silicon or other low energy material. The noted hydrophobic materials and associated base (e.g., photoresist) layers are set forth in U.S. application Ser. No. 10/880,701, which is incorporated by reference herein.

Microprojection members that can be employed with the present invention include, but are not limited to, the members disclosed in U.S. Pat. Nos. 6,083,196, 6,050,988 and 6,091,975, and Co-Pending Application No. 60/649,888, filed Jan. 31, 2005, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the Epoetin-based-base agent to be delivered can be contained in the hydrogel formulation disposed in a gel pack reservoir (discussed in detail below), contained in a biocompatible coating that is disposed on the microprojection member 30 or contained in both the hydrogel formulation and the biocompatible coating.

Referring now to FIG. 2, there is shown the microprojection member 30 having microprojections 34 that include a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39). In a preferred embodiment, the coating 35 covers each microprojection 34 in the range of approximately 75-90% of the overall length extending from the tip, as disclosed in Co-Pending Application No. 60/649,888, which is incorporated herein in its entirety.

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

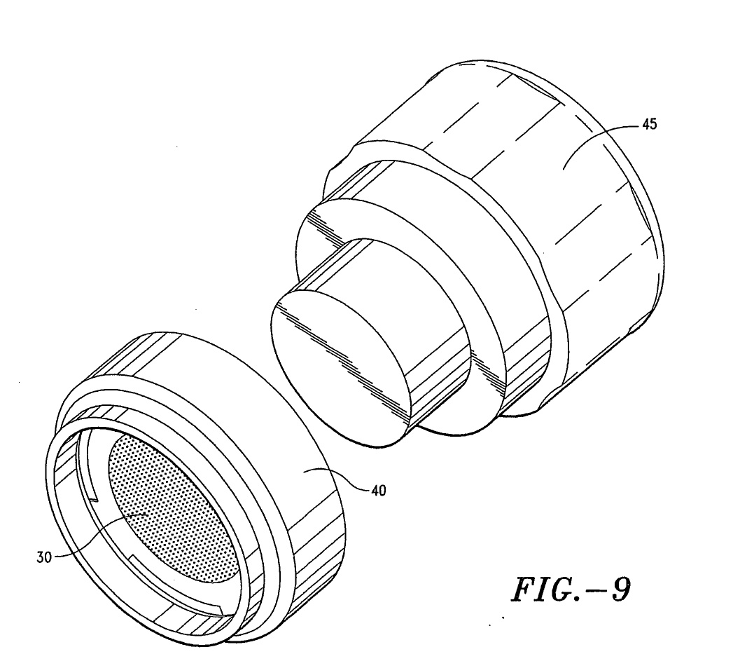

Referring now to FIGS. 7 and 8, for storage and application, the microprojection member 30 is preferably suspended in a retainer ring 40 by adhesive tabs 6, as described in detail in U.S. application Ser. No. 09/976,762 (Pub. No. 2002/0091357), which is incorporated by reference herein in its entirety.

After placement of the microprojection member 30 in the retainer ring 40, the microprojection member 30 is applied to the patient's skin. Preferably, the microprojection member 30 is applied to the patient's skin using an impact applicator 45, such as shown in FIG. 9 and described in Co-Pending U.S. application Ser. No. 09/976,978, which is incorporated by reference herein in its entirety.

As indicated, according to one embodiment of the invention, the coating formulations applied to the microprojection member 30 to form solid biocompatible coatings can comprise aqueous and non-aqueous formulations having at least one Epoetin-based agent. According to the invention, the Epoetin-based agent can be dissolved within a biocompatible carrier or suspended within the carrier.

In a preferred embodiment, the Epoetin-based agent is selected from the group consisting of recombinant Epoetin alpha, synthetic Epotein alpha, Epoetin alpha salts, simple derivatives and analogs of Epoetin alpha, recombinant Epoetin beta, synthetic Epotein beta, Epoetin beta salts, simple derivatives and analogs of Epoetin beta, darbepoetin alfa, recombinant darbepoetin alfa, synthetic darbepoetin alfa, darbepoetin alfa salts, simple derivatives and analogs of darbepoetin alfa, and closely related molecules of any of the foregoing.

Suitable Epoetin-based salts include, without limitation, acetate, propionate, butyrate, pentanoate, hexanoate, heptanoate, levulinate, chloride, bromide, citrate, succinate, maleate, glycolate gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate, sulfonate, tricarballylicate, malonate, adipate, citraconate, glutarate, itaconate, mesaconate, citramalate, dimethylolpropionate, tiglicate, glycerate, methacrylate, isocrotonate, β-hydroxybutyrate, crotonate, angelate, hydracrylate, ascorbate, aspartate and glutamate.

Suitable simple Epoetin-based derivatives and closely related molecules include, without limitation, pegylated derivatives, carbamylated derivatives, glycosylated derivatives, fusion derivatives, EPO muteins, nonhematopoietic mutants, and thrombopoietin.

Preferably, the Epoetin-based agent is present in the coating formulation in a range of approximately 15-30 wt. % of the coating formulation.

In one embodiment of the invention, the microprojection array is coated with a total dose of Epoetin-based agent in the range of approximately 15-120 μg.

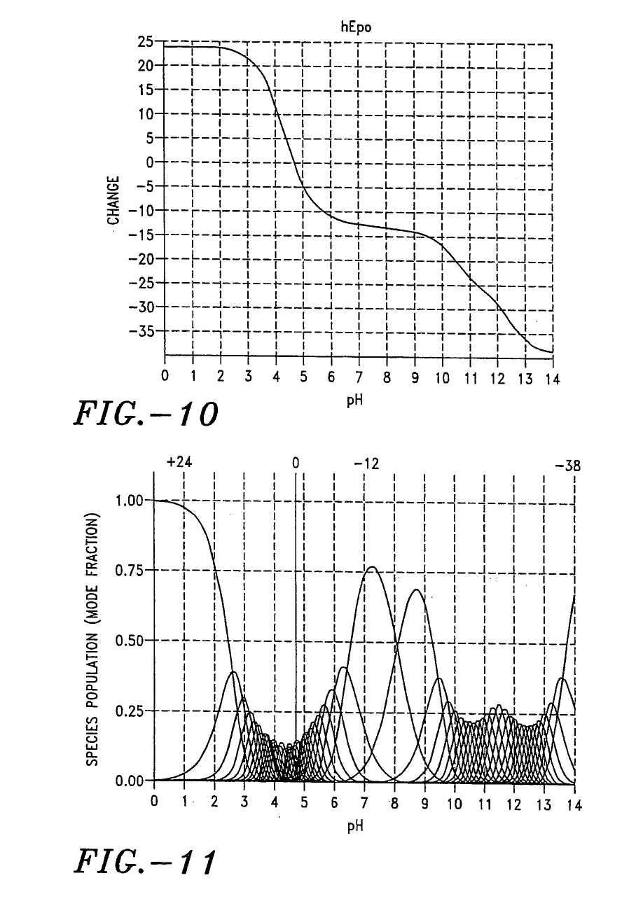

Referring now to FIG. 10, there is shown the predicted charge profile of an Epoetin-based agent. As is known in the art, Epoetin alpha, a 165 amino acid glycoprotein manufactured by recombinant DNA technology, has the same biological effects as endogenous EPO and contains the identical amino acid sequence of isolated natural EPO. Epoetin alpha has a molecular weight of 30,400 daltons and is produced by mammalian cells into which the human EPO gene has been introduced. Epoetin alpha presents thirty eight basic pKa and twenty-four acidic pKa. At pH 4.8, Epoetin alpha presents a zero net electric charge. This point is also called the isoelectric point or pI.

Referring now to FIG. 11, there is shown the predicted mole ratios of the net charged species of an Epoetin-based agent. As shown in FIG. 11, the neutral species only exist in significant amounts in the pH range pH 4.5 to pH 5.0. In this pH range, the peptide is expected to precipitate out of solution. Therefore, Epoetin agent solubility compatible with formulations suitable for the delivery system of the present invention is expected to be achieved at below about pH 4.5 or above about pH 5.0; more preferably below about pH 4.0 or above about pH 5.5.

Accordingly, in a preferred embodiment, the pH of the liquid formulation used to produce the biocompatible coating is below approximately pH 4.5 or above approximately pH 5.0. More preferably, the pH of the formulation used to produce the solid film is in the range of approximately pH 2 to pH 4.5, or in the range of approximately pH 5.0 to pH 11. Even more preferably, the pH of the liquid formulation used to produce the solid film is in the range of approximately pH 2 to pH 4, or in the range of approximately pH 5.5 to pH 9.5.

In one embodiment of the invention, the coating formulation includes at least one buffer. Suitable buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

In one embodiment of the invention, the coating formulation includes an antioxidant, which can comprise, without limitation, sequestering agents, such sodium citrate, citric acid, EDTA (ethylenedinitrilo-tetraacetic acid) or free radical scavengers, such as ascorbic acid, methionine, sodiumascorbate, and the like.

In one embodiment of the invention, the coating formulation includes at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

In one embodiment of the invention, the concentration of the surfactant is in the range of approximately 0.001-2 wt. % of the coating solution formulation.

In a further embodiment of the invention, the coating formulation includes at least one polymeric material or polymer that has amphiphilic properties. Examples of the noted polymers include, without limitation, cellulose derivatives, such as hydroxyethylcellulose (HEC), hydroxypropylmethylcellulose (HPMC), hydroxyl-propylcellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the polymer is in the range of approximately 0.1-5 wt. % of the coating formulation.

According to the invention, the coating formulation can further include a hydrophilic polymer. Preferably the hydrophilic polymer is selected from the following group: hydroxyethyl starch, dextran, poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 0.01-20 wt. %, more preferably, in the range of approximately 0.03-10 wt. % of the coating formulation. Even more preferably, the concentration of the hydrophilic polymer is in the range of approximately 0.1-5 wt. % of the coating formulation.

According to the invention, the coating formulation can further include a biocompatible carrier, such as those disclosed in Co-Pending U.S. application Ser. No. 10/127,108, which is incorporated by reference herein in its entirety. Examples of biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation.

In a further embodiment, the coating formulation includes at least one stabilizing agent, which can comprise, without limitation, a non-reducing sugar, a polysaccharide or a reducing sugar. Suitable non-reducing sugars for use in the methods and compositions of the invention include, for example, sucrose, trehalose, stachyose, or raffinose. Suitable polysaccharides for use in the methods and compositions of the invention include, for example, dextran, soluble starch, dextrin, and insulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

The coating formulations and, hence, biocompatible coatings of the invention can further include a vasoconstrictor, such as those disclosed in Co-Pending U.S. Patent Publication No. 2004/0115167 (Ser. No. 10/674,626,) which is incorporated by reference herein in its entirety. As set forth in the noted Co-Pending Application, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the coating formulations and, hence, solid biocompatible coatings of the invention (or the hydrogel formulations or solid film, discussed below) is particularly useful to prevent bleeding that can occur following application of the microprojection member or array and to prolong the pharmacokinetics of the Epoetin-based agent through reduction of the blood flow at the application site and reduction of the absorption rate from the skin site into the system circulation.

The concentration of the vasoconstrictor, if employed, is preferably in the range of approximately 0.1 wt. % to 10 wt. % of the coating formulation.

In yet another embodiment of the invention, the coating formulation includes at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride) and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilizing/complexing agent which can comprise Alpha-Cyclodextrin, Beta-Cyclodextrin, Gamma-Cyclodextrin, glucosyl-alpha-Cyclodextrin, maltosyl-alpha-Cyclodextrin, 2-hydroxypropyl-beta-Cyclodextrin,2-hydroxypropyl-gamma-Cyclo-dextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilizing/complexing agents are beta-cyclodextrin, hydroxypropyl beta-cyclodextrin, 2-hydroxypropyl-beta-Cyclodextrin and sulfobutylether7 beta-cyclodextrin.

The concentration of the solubilizing/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the range of approximately 1 wt. % to 50 wt. % of the coating formulation.

Other known formulation adjuvants can also be added to the coating formulations provided they do not adversely affect the necessary solubility and viscosity characteristics of the coating formulation and the physical integrity of the dried coating.

Preferably, the coating formulations have a viscosity less than approximately 500 centipoise and greater than approximately 3 centipose.

In one embodiment of the invention, the coating thickness is less than approximately 25 microns, more preferably, less than approximately 10 microns as measured from the microprojection surface.

The desired coating thickness is dependent upon several factors, including the required dosage and, hence, coating thickness necessary to deliver the dosage, the density of the microprojections per unit area of the sheet, the viscosity and concentration of the coating composition and the coating method chosen.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, lyophilized, freeze dried or similar techniques used to remove the water from the coating.

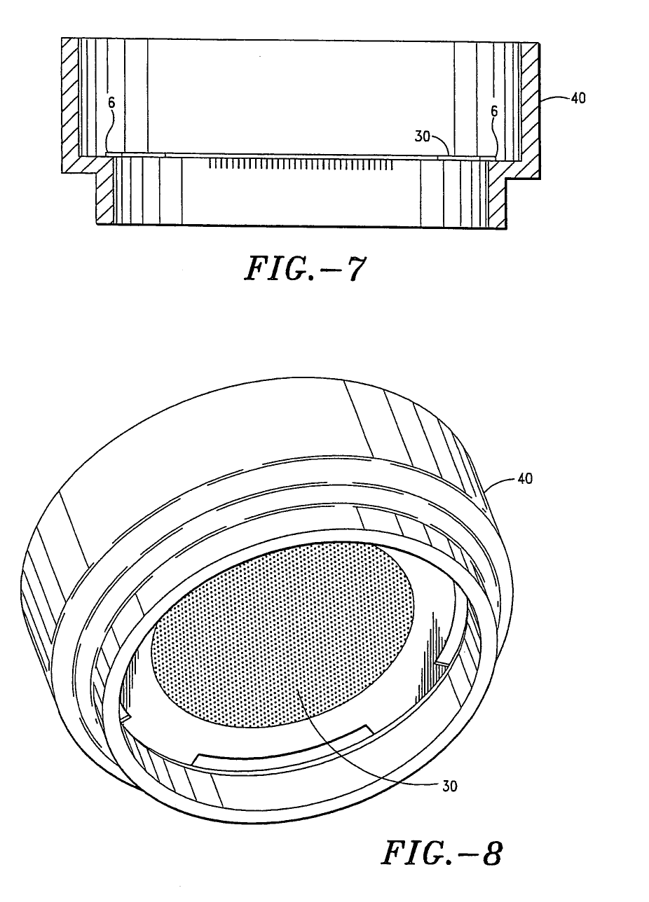

Referring now to FIG. 6, there is shown a further microprojection (or delivery) system (designated generally “80”) that can be employed within the scope of the present invention. As illustrated in FIG. 6, the system 60 includes a gel pack 62 and a microprojection assembly 70, having a microprojection member, such as the microprojection member 30 shown in FIG. 1.

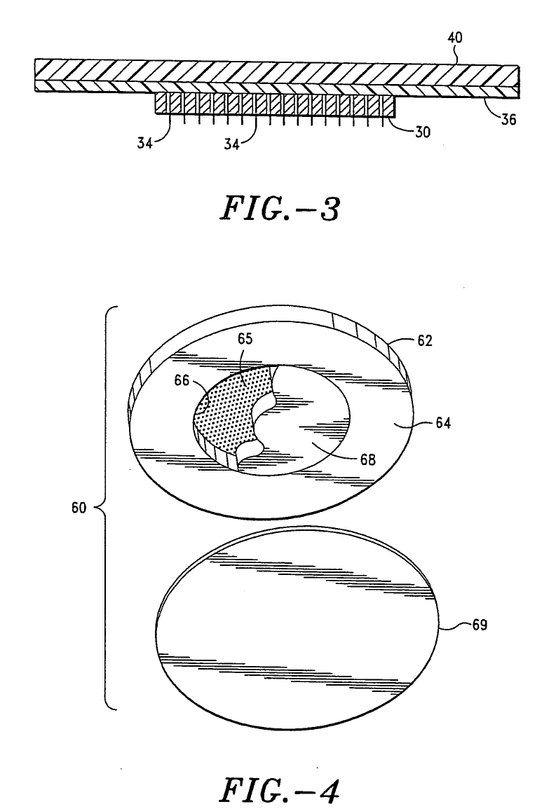

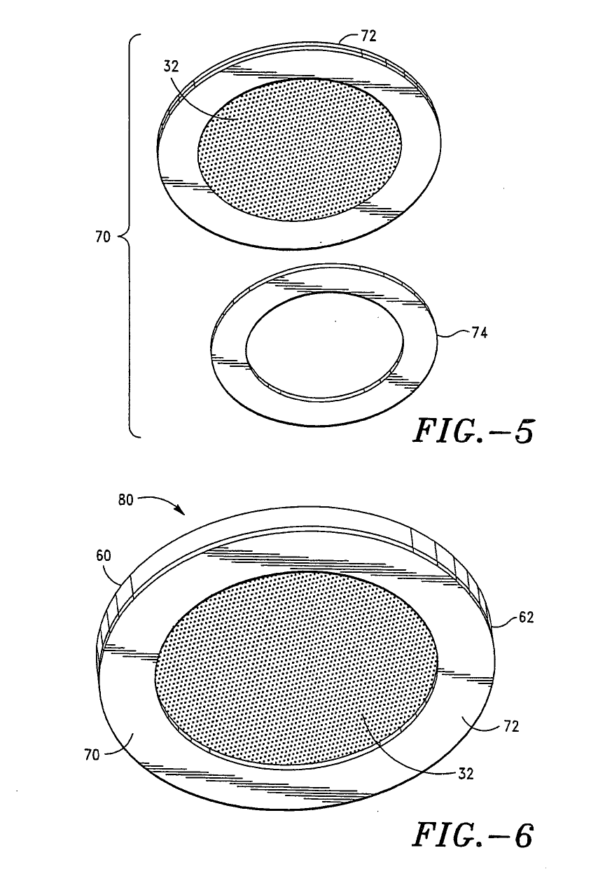

Referring now to FIG. 4, the gel pack 62 includes a housing or ring 64 having a centrally disposed reservoir or opening 66 that is adapted to receive a predetermined amount of a hydrogel formulation 68 therein. As illustrated in FIG. 4, the ring 64 further includes a backing member 65 that is disposed on the outer planar surface of the ring 64. Preferably, the backing member 65 is impermeable to the hydrogel formulation.

In a preferred embodiment, the gel pack 60 further includes a removable release liner 69 that is adhered to the outer surface of the gel pack ring 64 via a conventional adhesive. As described in detail below, the release liner 69 is removed prior to application of the gel pack 60 to the applied (or engaged) microprojection assembly 70.

Referring now to FIG. 5, the microprojection assembly 70 includes a backing membrane ring 72 and a similar microprojection array 32. The microprojection assembly further includes a skin adhesive ring 74.

Further details of the illustrated gel pack 60 and microprojection assembly 70, as well as additional embodiments thereof that can be employed within the scope of the present invention are set forth in Co-Pending application Ser. No. 10/971,430, which is incorporated by reference herein in its entirety.

As indicated above, in at least one embodiment of the invention, the hydrogel formulation contains at least one Epoetin-based agent. In an alternative embodiment of the invention, the hydrogel formulation is devoid of an Epoetin-based agent and, hence, is merely a hydration mechanism.

According to the invention, when the hydrogel formulation is devoid of an Epoetin-based agent, the Epoetin-based agent is either coated on the microprojection array 32, as described above, or contained in a solid film, such as disclosed in PCT Pub. No. WO 98/28037, which is similarly incorporated by reference herein in its entirety, on the skin side of the microprojection array 32, such as disclosed in the noted Co-Pending application Ser. No. 10/971,430 or the top surface of the array 32.

Preferably, the hydrogel formulations of the invention comprise water-based hydrogels. Hydrogels are preferred formulations because of their high water content and biocompatibility.

As is well known in the art, hydrogels are macromolecular polymeric networks that are swollen in water. Examples of suitable polymeric networks include, without limitation, hydroxyethyl starch, hydroxyethylcellulose (HEC), hydroxypropylmethylcellulose (HPMC), hydroxypropylcellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), dextran and pluronics. The most preferred polymeric materials are cellulose derivatives. These polymers can be obtained in various grades presenting different average molecular weight and therefore exhibit different rheological properties.

Preferably, the concentration of the polymeric material is in the range of approximately 0.5-40 wt. % of the hydrogel formulation.

The hydrogel formulations of the invention preferably have sufficient surface activity to insure that the formulations exhibit adequate wetting characteristics, which are important for establishing optimum contact between the formulation and the microprojection array and skin and, optionally, the solid film.

According to the invention, adequate wetting properties are achieved by incorporating a wetting agent, such as a surfactant or polymeric material having amphiphilic properties, in the hydrogel formulation. Optionally, a wetting agent can also be incorporated in the solid film.

According to the invention, the surfactant(s) can be zwitterionic, amphoteric, cationic, anionic, or nonionic. Examples of suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laureate, and alkoxylated alcohols such as laureth-4. Most preferred surfactants include Tween 20, Tween 80, and SDS.

Preferably, the concentration of the surfactant is in the range of approximately 0.001-2 wt. % of the hydrogel formulation. The concentration of the polymer that exhibits amphiphilic properties is preferably in the range of approximately 0.5-40 wt. % of the hydrogel formulation.

As will be appreciated by one having ordinary skill in the art, the noted wetting agents, surfactants or polymeric materials can be used separately or in combinations.

In a further embodiment of the invention, the hydrogel formulation includes at least one of the aforementioned solubilizing/complexing agents.

According to the invention, the hydrogel formulation can similarly include at least one of the pathway patency modulators disclosed in Co-Pending U.S. application Ser. No. 09/950,436.

The hydrogel formulation can further include at least one of the aforementioned vasoconstrictors.

In another embodiment, the hydrogel formulation includes at least one of the aforementioned stabilizing agents, which can similarly comprise a non-reducing sugar, a polysaccharide or a reducing sugar.

In one embodiment of the invention, the hydrogel formulation includes one of the aforementioned antioxidants.

In one embodiment of the invention, the hydrogel formulation includes at least one of the aforementioned buffers.

In another embodiment of the invention, the hydrogel formulation includes at least one non-aqueous solvent, such as ethanol, isopropanol, methanol, propanol, butanol, propylene glycol, dimethyl sulphoxide and polyethylene glycol 400. Preferably, the non-aqueous solvent is present in the range of approximately 1 wt. % to 50 wt. % of the hydrogel formulation.

The hydrogel formulations of the invention exhibit adequate viscosity so that the formulation can be contained in the gel pack 60, keeps its integrity during the application process, and is fluid enough so that it can flow through the microprojection assembly openings and into the skin pathways.

For hydrogel formulations that exhibit Newtonian properties, the viscosity of the hydrogel formulation is preferably in the range of approximately 2-300 Poises (P), as measured at 25° C. For shear-thinning hydrogel formulations, the viscosity, as measured at 25° C., is preferably in the range of 1.5-30 P or 0.5 and 10 P, at shear-rates of 667/s and 2667/s, respectively. For dilatant formulations, the viscosity, as measured at 25° C., is preferably in the range of approximately 1.5-30 P, at a shear rate of 667/s.

As indicated, in at least one embodiment of the invention, the hydrogel formulation contains at least one Epoetin-based agent. According to the invention, when the hydrogel formulation contains one of the aforementioned Epoetin-based agents, the Epoetin-based agent can be present at a concentration in excess of saturation or below saturation. The amount of Epoetin-based agent employed in the microprojection system will be that amount necessary to deliver a therapeutically effective amount of the Epoetin-based agent to achieve the desired result. In practice, this will vary widely depending upon the particular Epoetin-based agent, the site of delivery, the severity of the condition, and the desired therapeutic effect.

In one embodiment of the invention, the concentration of the Epoetin-based agent is in the range of at least 0.1-2 wt. % of the hydrogel formulation.

Preferably, the total dose of Epoetin-based agent delivered intracutaneously per administration is in the range of approximately 10-200 μg. More preferably, the total dose of Epoetin-based agent delivered intracutaneously per administration is in the range of about 15-150 μg delivered once every two weeks and up to once a day.

In accordance with yet another embodiment of the invention, the microprojection system for delivering a Epoetin-based agent comprises (i) a gel pack containing a hydrogel formulation and (ii) a microprojection member having top and bottom surfaces, a plurality of openings that extend through the microprojection member and a plurality of stratum corneum-piercing microprotrusions that project from the bottom surface of the microprojection member, the microprojection member including a solid film having at least one Epoetin-based agent. Details of the noted system are set forth in Co-Pending application Ser. No. 10/971,430, which is incorporated by reference herein in its entirety.

In accordance with one embodiment of the invention, the solid film is disposed proximate the top surface of the microprojection member. In another embodiment, the solid film is disposed proximate the bottom surface of the microprojection member.

In one embodiment, the hydrogel formulation includes at least one Epoetin-based agent.

In another embodiment, the hydrogel formulation is devoid of an Epoetin-based agent.

In one embodiment, the solid film is made by casting a liquid formulation consisting of the Epoetin-based agent, a polymeric material, such as hyroxyethyl starch, dextran, hydroxyethylcellulose (HEC), hydroxypropylmethylcellulose (HPMC), hydroxypropylcellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone), or pluronics, a plasticising agent, such as glycerol, propylene glycol, or polyethylene glycol, a surfactant, such as Tween 20 or Tween 80, and a volatile solvent, such as water, isopropanol, methanol or ethanol.

In one embodiment, the liquid formulation used to produce the solid film comprises 0.1-20 wt. % Epoetin-based agent, 5-40 wt. % polymer, 5-40 wt. % plasticiser, 0-2 wt. % surfactant, and the balance of volatile solvent. Following casting and subsequent evaporation of the solvent, a solid film is produced.

Preferably, the pH of the liquid formulation used to produce the solid film is below approximately pH 4.5 or above approximately pH 5.0. More preferably, the pH of the formulation used to produce the solid film is in the range of approximately pH 2 to pH 4.5, or in the range of approximately pH 5.0 to pH 11. Even more preferably, the pH of the liquid formulation used to produce the solid film is in the range of approximately pH 2 to pH 4, or in the range of approximately pH 5.5 to pH 9.5.

In another embodiment, the liquid formulation used to produce the solid film includes at least one of the aforementioned stabilizing agents.

In one embodiment of the invention, the liquid formulation used to produce the solid film includes at least one of the aforementioned buffers.

In another embodiment of the invention, the liquid formulation used to produce the solid film includes at least one of the aforementioned solubilizing/complexing agents.

In one embodiment of the invention, the liquid formulation used to produce the solid film includes at least one of the aforementioned antioxidants.

In a further embodiment of the invention, the liquid formulation used to produce the solid film includes at least one of the aforementioned vasoconstrictors.

In a further embodiment of the invention, the liquid formulation used to produce the solid film includes at least one of the aforementioned pathway patency modulators.

As stated above, Epoetin agent solubility compatible with formulations suitable for the delivery system of the present invention is expected to be achieved at below about pH 4.5 or above about pH 5.0. Accordingly, each of the coating, hydrogel and solid film formulation embodiments described herein can include a counterion or counterion mixture appropriate to the Epoetin agent and desired formulation pH range.

In one embodiment of the invention, the acidic counterion comprises a non-volatile weak acid. Non-volatile weak acid counterions are defined as weak acids presenting at least one acidic pKa and a melting point higher than about 50° C. or a boiling point higher than about 170° C. at Pₐₜₘ. Examples of such acids include citric acid, succinic acid, glycolic acid, lactic acid, malic acid, pyruvic acid, tartaric acid, tartronic acid, and fumaric acid.

In another embodiment of the invention, the counterion comprises a strong acid. Strong acids are defined as presenting at least one pKa lower than about 2. Examples of such acids include hydrochloric acid, hydrobromic acid, nitric acid, sulfuric acid, maleic acid, phosphoric acid, benzene sulfonic acid and methane sulfonic acid.

In another embodiment of the invention, the counterion comprises a mixture of counterions, wherein at least one of the counterions comprises a strong acid and at least one of the counterions comprises a non-volatile weak acid.

In yet another embodiment of the invention, the counterion comprises a mixture of counterions, wherein at least one of the counterions comprises a strong acid and at least one of the counterions comprises a weak acid with high volatility. Highly volatile weak acid counterions are defined as weak acids presenting at least one pKa higher than about 2 and having a melting point lower than about 50° C. or a boiling point lower than about 170° C. at Pₐₜₘ. Examples of such acids include acetic acid, propionic acid, pentanoic acid and the like.

In another embodiment of the invention, the basic counterion comprises a weak base with low volatility. Low volatility weak base counterions are defined as weak bases presenting at least one basic pKa and a melting point higher than about 50° C. or a boiling point higher than about 170° C. at Pₐₜₘ. Examples of such bases include monoethanolomine, diethanolamine, triethanolamine, tromethamine, methylglucamine, glucosamine.

In another embodiment of the invention, the counterion comprises a strong base presenting at least one pKa higher than about 12. Examples of such bases include sodium hydroxide, potassium hydroxide, calcium hydroxide, and magnesium hydroxide.

Another embodiment of the invention is directed to a mixture of counterions, wherein at least one of the counterions comprises a strong base and at least one of the counterions comprises a weak base with low volatility.

Another embodiment of the invention is directed to a mixture of counterions, wherein at least one of the counterions comprises a strong base and at least one of the counterions comprises a weak base with high volatility. Highly volatile weak base counterions are defined as weak bases presenting at least one pKa lower than about 12 and a melting point lower than about 50° C. or a boiling point lower than about 170° C. at Pₐₜₘ. Examples of such bases include ammonia and morpholine.

In accordance with one embodiment of the invention, the method for delivering an Epoetin-based agent to a patient includes the following steps: (i) providing a microprojection member (e.g., 30) having a biocompatible coating that includes at least one Epoetin-based agent and (ii) applying the coated microprojection member to the patient's skin, wherein the microprojections pierce the stratum corneum. The coated microprojection member is preferably left on the skin for a period lasting from 5 seconds to 24 hours. Following the desired wearing time, the microprojection member is removed.

In accordance with a further embodiment of the invention, the method for delivering an Epoetin-based agent to a patient includes the following steps: (i) providing a microprojection member (e.g., 30) having a solid film disposed proximate to (or on) the member, the film including at least one Epoetin-based agent and (ii) applying the microprojection member to the patient's skin, wherein the microprojections pierce the stratum corneum. The microprojection member is preferably left on the skin for a period lasting from 5 minutes to 24 hours. Following the desired wearing time, the microprojection member is removed.

In another embodiment of the invention, the microprojection assembly 70 is applied to the patient's skin. After application of the microprojection assembly 70, the release liner 69 is removed from the gel pack 60. The gel pack 60 is then placed on the microprojection assembly 70, whereby the hydrogel formulation 68 is released from the gel pack 60 through the openings 38 in the microprojection array 32, passes through the microslits in the stratum corneum formed by the microprojections 34, migrates down the outer surfaces of the microprojections 34 and through the stratum corneum to achieve local or systemic therapy.

Preferably, the gel pack 60 is left on the patient's skin for a period in the range of approximately 5 min to 24 hours. Following the desired wearing time, the gel pack 60 and microprojection assembly 70 are removed from the skin.

In one embodiment of the invention, the microprojection assembly 70 includes a microprojection array 34 having a biocompatible coating disposed thereon that includes at least one Epoetin-based agent, as illustrated in FIG. 2.

In another embodiment, the Epoetin-based agent is contained in a hydrogel formulation in the gel pack 60.

In a further embodiment, the Epoetin-based agent is contained in a hydrogel formulation in the gel pack 60 and in a biocompatible coating applied to the microprojection assembly 70.

According to a further embodiment of the invention, the microprojection assembly 70 is applied to the patient's skin and immediately removed. The release liner 69 is then removed from the gel pack 60 and the gel pack 60 is placed on the pretreated skin, whereby the hydrogel formulation 68 is released from the gel pack 60 and passes through the microslits in the stratum corneum formed by the microprojections 34.

Preferably, the gel pack 60 is left on the patient's skin for a period in the range of approximately 5 min to 24 hours. Following the desired wearing time, the gel pack 60 is removed from the skin.

In the noted embodiment, the Epoetin-based agent is contained in the hydrogel formulation in the gel pack 60.

In any of the foregoing embodiments, the total dose of Epoetin-based agent delivered intracutaneously per administration is in the range of approximately 10-200 μg. More preferably, the total dose of Epoetin-based agent delivered intracutaneously per administration is in the range of about 15-150 μg delivered once every two weeks and up to once a day.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body can be achieved in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods such as sonophoresis or piezoelectric devices can be used in conjunction with the invention.

When the invention is employed in conjunction with electrotransport, sonophoresis or piezoelectric systems, the microprojection assembly 70 is first applied to the skin as explained above. The release liner 69 is removed from the gel pack 60, which is part of the electrotransport, sonophoresis or piezoelectric system. This assembly is then placed on the skin template, whereby the hydrogel formulation 68 is released from the gel pack 60 and passes through the microslits in the stratum corneum formed by the microprojections 34 to achieve local or systemic therapy with additional facilitation of drug transport via the electrotransport, sonophoresis or piezoelectric processes. When the invention is employed in conjunction with one of the noted systems, the total skin contact area can be in the range of approximately 2-120 cm².

### EXAMPLES

The following examples are given to enable those skilled in the art to more clearly understand and practice the present invention. They should not be considered as limiting the scope of the invention but merely as being illustrated as representative thereof.

### Example 1

Delivery Efficacy: a 2.5 wt. % Epoetin alpha solution having a pH of approximately pH 7.0 is prepared. 0.1 wt. % hydroxyethyl cellulose and 0.2 wt. % of the surfactant Tween 20 is then added. The coating solution is then applied to the microprojections using the coating methods described in U.S. Pat. Pub. No. 2002/0132054. The coating is evaluated and found to be well distributed across the projections. The coated and dried projections of a 2 cm² device are found to contain 60 μg of Epoetin-based agent. When the device is applied to the skin of a subject and maintained in contact thereto for a duration of 5 min, delivery of more than 80% of the Epoetin-based agent contained on the projections is achieved.

### Example 2

Pharmacokinetic Evaluation: A hydrogel formulation comprising 1 wt. % EPO, 2 wt. % hydroxyethyl cellulose, and 0.2 wt. % of the surfactant Tween 20 in water at pH 7 is prepared. Pathway patency modulators are also present in the formulation. The microprojection member is then applied to the skin of a subject (as described in Co-Pending application Ser. No. 10/971,430, for a period of 24 hours. Following application, blood samples are taken at various times and evaluated for Epoetin-based content. Pharmacokinetic assessment indicates delivery of about 150 μg over the 24-hour application time.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A delivery system for transdermally delivering an Epoetin-based agent to a patient, comprising:
a microprojection member having a plurality of microprojections that are adapted to pierce the stratum corneum of the patient; and
a biocompatible coating disposed on said microprojection member, said coating being formed from a coating formulation having at least one Epoetin-based agent disposed therein.

2. The delivery system of claim 1, wherein said coating is disposed on at least one of said plurality of microprojections.

3. The delivery system of claim 1, wherein said coating formulation comprises an aqueous formulation.

4. The delivery system of claim 1, wherein said coating formulation comprises a non-aqueous formulation.

5. The delivery system of claim 1, wherein said Epoetin-based agent is selected from the group consisting of Epoetin alpha, Epoetin beta, darbepoetin alfa, and pharmaceutically acceptable salts, analogs, simple derivatives, closely related molecules and combinations thereof.

6. The delivery system of claim 5, wherein said Epoetin-based agent comprises Epoetin alpha.

7. The delivery system of claim 1, wherein said Epoetin-based agent comprises in the range of approximately 1-30 wt. % of said coating formulation.

8. The delivery system of claim 1, wherein the coating on the microprojection member comprises a dose of Epoetin-based agent in the range of about 15 to 200 μg.

9. The delivery system of claim 1, wherein the pH of said coating formulation is below approximately pH 4.5 or above approximately pH 5.

10. The delivery system of claim 1, wherein said coating formulation includes at least one buffer selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarbally acid, malonic acid, adipic acid, citraconic acid, glutaratic acid, itaconic acid, mesaconic acid, citramalic acid, dimethylopropionic acid, tiglic acid, glyceric acid, methacrylic acid, isocrotonic acid, 0-hydroxybutyric acid, crotonic acid, angelic acid, hydracrylic acid, aspartic acid, glutamic acid, glycine and mixtures thereof.

11. The delivery system of claim 1, wherein said coating formulation includes at least one surfactant selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, sorbitan derivatives, alkoxylated alcohols and mixtures thereof.

12. The delivery device of claim 1, wherein said coating formulation includes at least one counterion to neutralize the charge of the Epoetin-based agent.

13. The delivery system of claim 1, wherein said coating formulation includes a hydrophilic polymer selected from the following group consisting of hydroxyethyl starch, dextran, poly(vinyl alcohol), poly(ethylene oxide), poly(2-hydroxyethyl-methacrylate), poly(n-vinyl pyrolidone), polyethylene glycol and mixtures thereof.

14. The delivery system of claim 1, wherein said coating formulation includes a biocompatible carrier selected from the group consisting of human albumin, bioengineered human albumin, polyglutamic acid, polyaspartic acid, polyhistidine, pentosan polysulfate, polyamino acids, sucrose, trehalose, melezitose, raffinose, stachyose, mannitol and like sugar alcohols.

15. The delivery system of claim 1, wherein said coating formulation includes a stabilizing agent selected from the group consisting of a non-reducing sugar, a polysaccharide and a reducing sugar.

16. The delivery system of claim 1, wherein said coating formulation includes at least one vasoconstrictor selected from the group consisting of amidephrine, cafaminol, cyclopentaimine, deoxyepinephrine, epinephrine, felypressin, indanzoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline, and mixtures thereof.

17. The delivery system of claim 1, wherein said coating formulation includes at least one pathway patency modulator selected from the group consisting of osmotic agents, zwitterionic compounds, anti-inflammatory agents and anticoagulants.

18. The delivery system of claim 1, wherein said coating formulation includes a solubilising/complexing agent selected from the group consisting of Alpha-Cyclodextrin, Beta-Cyclodextrin, Gamma-Cyclodextrin, glucosyl-alpha-Cyclodextrin, maltosyl-alpha-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin.

19. A delivery system for transdermally delivering a Epoetin-based agent to a patient, comprising:
a microprojection member having a plurality of microprojections that are adapted to pierce the stratum corneum of the patient; and
a hydrogel formulation having at least one Epoetin-based agent, said hydrogel formulation being in communication with said microprojection member.

20. The delivery system of claim 19, wherein said Epoetin-based agent comprises in the range of approximately 1 to 30 wt. % of the hydrogel formulation.

21. The delivery system of claim 19, wherein said Epoetin-based agent is selected from the group consisting of Epoetin alpha, Epoetin beta, darbepoetin alfa, and pharmaceutically acceptable salts, analogs, simple derivatives, closely related molecules and combinations thereof.

22. The delivery system of claim 19, wherein said Epoetin-based agent comprises Epoetin alpha.

23. The delivery system of claim 19, wherein the pH of said hydrogel formulation is below approximately pH 4.5 or above approximately pH5.

24. The delivery system of claim 19, wherein said hydrogel formulation comprises a water-based hydrogel having a macromolecular polymeric network.

25. The delivery system of claim 19, wherein said hydrogel formulation includes at least one surfactant, selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, sorbitan derivatives, and alkoxylated alcohols.

26. A delivery system for transdermally delivering a Epoetin-based agent to a patient; comprising:
a microprojection member having a plurality of microprojections that are adapted to pierce the stratum corneum of the patient;
a solid state formulation disposed proximate said microprojection member; and
a hydrogel formulation, said hydrogel formulation adapted to communicate with said solid state formulation.

27. The delivery system of claim 26, wherein said solid state formulation is a solid film made by casting a liquid formulation comprising at least one Epoetin-based agent, a polymeric material, a plasticizing agent, a surfactant and a volatile solvent.

28. The delivery system of claim 27, wherein said liquid formulation comprises 1-30 wt. % Epoetin-based agent, 5-40 wt. % polymer, 5-40 wt. % plasticizer, 0-2 wt. % surfactant, and the balance comprising volatile solvent.

29. The delivery system of claim 26, wherein the pH of said liquid formulation is below approximately pH4.5 or above approximately pH 5.

30. A method of transdermally delivering a Epoetin-based agent to a patient, comprising the steps of:
providing a microprojection member having a plurality of microprojections, said microprojection member having a coating disposed thereon, said coating including at least one Epoetin-based agent;
applying said microprojection member to a skin site of said patient, whereby said plurality of microprojections pierce the stratum corneum and deliver said Epoetin-based agent to said patient; and
removing said microprojection member from said skin site.

31. The method of claim 30, wherein said microprojection member remains applied to said skin site for a period of time in the range of 5 sec. to 24 hrs.

32. The method of claim 30, wherein said Epoetin-based agent is selected from the group consisting of Epoetin alpha, Epoetin beta, darbepoetin alfa, and pharmaceutically acceptable salts, analogs, simple derivatives, closely related molecules and combinations thereof.

33. The method of claim 30, wherein said Epoetin-based agent comprises Epoetin alpha.

34. The method of claim 30, wherein said Epoetin-based agent comprises in the range of approximately 15 μg-200 μg of said biocompatible coating.

35. The method of claim 30, wherein said delivery of said Epoetin-based agent exhibits improved pharmacokinetics compared to the pharmacokinetic characteristics of subcutaneous delivery.

36. A method for transdermally delivering a Epoetin-based agent to a patient, comprising the steps of:
providing a microprojection assembly having a microprojection member and a gel pack, said microprojection member including a plurality of microprojections, said gel pack including a hydrogel formulation having at least one Epoetin-based agent;
applying said microprojection member to a skin site of said patient, whereby a plurality of microslits are formed in the patient's stratum-corneum;
placing said gel pack on said microprojection member, whereby said hydrogel formulation is released from said gel pack and migrates into and through said microslits formed by said microprojections; and
removing said microprojection member from said skin site.

37. The method of claim 36, wherein said gel pack includes a release liner and said method includes the step of removing said release liner prior to placing said gel pack on said microprojection member.

38. The method of claim 36, wherein said microprojection member includes a biocompatible coating having at least one Epoetin-based agent.

39. The method of claim 36, wherein said microprojection member remains applied to said skin site for a period of time in the range of 5 min. to 24 hrs.

40. The method of claim 36, wherein said Epoetin-based agent is selected from the group consisting of Epoetin alpha, Epoetin beta, darbepoetin alfa, and pharmaceutically acceptable salts, analogs, simple derivatives, closely related molecules and combinations thereof.

41. The method of claim 36, wherein said Epoetin-based agent comprises Epoetin alpha.

42. The method of claim 36, wherein said Epoetin-based agent comprises in the range of approximately 1-30 wt. % of said hydrogel formulation.

43. The method of claim 36, wherein said delivery of said Epoetin-based agent exhibits improved pharmacokinetics compared to the pharmacokinetic characteristics of subcutaneous delivery.

44. A method for transdermally delivering a Epoetin-based agent to a patient, comprising the steps of:
providing a microprojection assembly having a microprojection member and a gel pack, said microprojection member including a plurality microprojections, said microprojection member further including a biocompatible coating having at least one Epoetin-based agent, said gel pack including a hydrogel formulation;
applying said microprojection member to a skin site of said patient, whereby a plurality of microslits are formed in the patient's stratum-corneum;
placing said gel pack on said microprojection member, whereby said hydrogel formulation is released from said gel pack and migrates into and through said microslits formed by said microprojections; and
removing said microprojection member from said skin site.

45. The method of claim 44, wherein said gel pack includes a release liner and said method includes the step of removing said release liner prior to placing said gel pack on said microprojection member.

46. The method of claim 44, wherein said microprojection member remains applied to said skin site for a period of time in the range of 5 min. to 24 hrs.

47. The method of claim 44, wherein said Epoetin-based agent is selected from the group consisting of Epoetin alpha, Epoetin beta, darbepoetin alfa, and pharmaceutically acceptable salts, analogs, simple derivatives, closely related molecules and combinations thereof.

48. The method of claim 44, wherein said Epoetin-based agent comprises in the range of approximately 15 μg-200 μg of said biocompatible coating.

49. The method of claim 44, wherein said Epoetin-based agent comprises Epoetin alpha.

50. The method of claim 44, wherein said delivery of said Epoetin-based agent exhibits improved pharmacokinetics compared to pharmacokinetics characteristic of subcutaneous delivery.

51. A method for transdermally delivering a Epoetin-based agent to a patient, comprising the steps of:
providing a microprojection assembly having a microprojection member, a gel pack and a solid state formulation, said microprojection member including a plurality of microprojections, said gel pack including a hydrogel formulation, said solid state formulation being disposed proximate said microprojection member and including at least one Epoetin-based agent;
applying said microprojection member to a skin site of said patient, whereby a plurality of microslits are formed in the patient's stratum-corneum;
placing said gel pack on said microprojection member, whereby said hydrogel formulation is released from said gel pack and migrates into and through said microslits formed by said microprojections; and
removing said microprojection member from said skin site.

52. The method of claim 51, wherein said gel pack includes a release liner and said method includes the step of removing said release liner prior to placing said gel pack on said microprojection member.

53. The method of claim 51, wherein said microprojection member remains applied to said skin site for a period of time in the range of 5 min. to 24 hrs.

54. The method of claim 51, wherein said Epoetin-based agent is selected from the group consisting of Epoetin alpha, Epoetin beta, darbepoetin alfa, and pharmaceutically acceptable salts, analogs, simple derivatives, closely related molecules and combinations thereof.

55. The method of claim 51, wherein said solid state formulation is formed from a liquid formulation having in the range of about 1-30 wt. % of said Epoetin-based agent.

56. The method of claim 51, wherein said Epoetin-based agent comprises Epoetin alpha.

57. The method of claim 51, wherein said delivery of said Epoetin-based agent exhibits improved pharmacokinetics compared to the pharmacokinetic characteristics of subcutaneous delivery.
